(19) United States
(12) Patent Application Publication
(10) Pub. No.: US 2024/0055139 A1
(43) Pub. Date: Feb. 15, 2024

(54) DISEASE OUTBREAK FORECASTING APPARATUS, AND METHOD THEREOF AND STORAGE MEDIUM

(71) Applicant: OSAKA UNIVERSITY, Osaka (JP)

(72) Inventors: Ken NAKATA, Osaka (JP); Yasushi SAKATA, Osaka (JP); Keita YAMASAKI, Osaka (JP); Teruki YOKOYAMA, Osaka (JP); Shoji KONDA, Osaka (JP)

(21) Appl. No.: 18/030,990

(22) PCT Filed: Oct. 12, 2021
(86) PCT No.: PCT/JP2021/037665

§ 371 Date: Sep. 25, 2023

(30) Foreign Application Priority Data
Oct. 12, 2020 (JP) 2020-171618

(51) Int. Cl. G16H 50/80; A61B 5/0205; A61B 5/024; A61B 5/11

(52) U.S. Cl. CPC G16H 50/80; A61B 5/02055; A61B 5/024; A61B 5/11

(57) ABSTRACT

An apparatus (1) includes an exercise sensor (31) that continuously detects a movement of an exerciser accompanied by intermittent exercise, and a heart rate meter (32) that continuously measures a heart rate of the exerciser, and further includes an activity detection unit (12), a heart rate monitoring unit (13), an activity monitoring unit (14), and a notification processing unit (15). The activity detection unit (12) detects, from the movement of the exerciser, an activity intensity, a high activity intensity period, and a low activity intensity period. The heart rate monitoring unit (13) detects a declining trend in the heart rate of the exerciser, in the low activity intensity period each time, and monitors whether or not the declining trend of this time is low as compared with a predetermined reference. The activity monitoring unit (14), in a case in which the monitoring by the heart rate monitoring unit is affirmed, monitors whether or not the activity intensity to be detected in the high activity intensity period of a next time is low as compared with activity intensities detected in high activity intensity periods up to a previous time. The notification processing unit (15), in a case in which the monitoring by the activity monitoring unit is affirmed, forecasts an outbreak of disease. With this configuration, the outbreak of disease in an exerciser during an exercise is forecasted with higher accuracy.

### TECHNICAL FIELD

The present invention relates to techniques of forecasting an outbreak of disease during exercise or sport accompanied by intermittent activity, by integrating medical knowledge to biological information obtained by sensing.

### BACKGROUND ART

As a serious accident during exercise or sporting activity, sudden death from ventricular fibrillation, death due to heatstroke, or the like has been known. These can occur not only in a patient who has an internal disease, or an elderly person, but in a healthy younger person, and prevention is difficult to achieve only by a medical checkup performed prior to start of the exercise or the sporting activity. In recent years, a method of monitoring a heart rate during exercise or sporting activity by use of a wearable sensor such as a wrist watch type and a wear type, has been known.

Patent Literature 1 discloses an onset risk determination apparatus that, by comparing a pulse rate and a reference pulse rate being a reference value, determines the risk of the onset of heatstroke according to a difference between the pulse rate and the reference pulse rate, during outdoor work, and notifies a user of the determined risk of the onset of heat stroke and subsequently estimates whether or not the user takes evacuation action compared with activity intensity.

### CITATION LIST

### Patent Literature

[Patent Literature 1] WO2016/203622

### SUMMARY OF INVENTION

### Technical Problem

The above heart rate monitoring method is primarily intended for heart beat monitoring (% HRmax or the like) as an index of training intensity, and a case applied to forecasting of disease or fatigue based on medical knowledge is not seen. Although forecasting of fatigue or disease from an activity level and the heart beat monitoring during exercise or sporting activity of not only a late middle-aged and elderly person but also a younger person or an early middle-aged person is an important issue, no sufficient solution has not yet been proposed in the present situation. In addition, although a method of electrically monitoring the function of a heart as an electrocardiogram by attaching an electrode to a body surface or an apparatus that senses a pulse rate by use of a pulse pressure or a light beam has been known, it is difficult to monitor electrocardio or a pulse rate with high accuracy, during exercise.

In addition, although the onset risk determination apparatus disclosed in Patent Literature 1 uses a pulse rate and activity intensity of a user, the activity intensity of a user is used to estimate whether or not the user has taken the own evacuation action after being notified of the risk of onset, and is not used to make a determination of whether or not the risk occurs.

In view of the foregoing, the present invention provides a disease outbreak forecasting apparatus that forecasts an outbreak of disease from a changing state of a heart rate and activity intensity during exercise to be performed intermittently, and a method thereof and a storage medium.

### Solution to Problem

A disease outbreak forecasting apparatus according to the present invention includes an exercise sensor that continuously detects a movement of an exerciser accompanied by intermittent exercise, a heart rate meter that continuously measures a heart rate of the exerciser, an activity detection unit that detects, from the movement of the exerciser that is detected by the exercise sensor, an activity intensity, a high activity intensity period, and a low activity intensity period, a first monitoring unit that detects a declining trend in the heart rate of the exerciser, in the low activity intensity period each time, and monitors whether or not the declining trend of this time is low as compared with a predetermined reference, a second monitoring unit that, in a case in which the monitoring by the first monitoring unit is affirmed, monitors whether or not the activity intensity to be detected in the high activity intensity period of a next time is low as compared with activity intensities detected in high activity intensity periods up to a previous time, and a notifying unit that, in a case in which the monitoring by the second monitoring unit is affirmed, forecasts an outbreak of disease.

In addition, a disease outbreak forecasting method according to the present invention includes continuously detecting a movement of an exerciser accompanied by intermittent exercise by an exercise sensor, and continuously measuring a heart rate of the exerciser by a heart rate meter, detecting, from the movement of the exerciser that is detected by the exercise sensor, an activity intensity, a high activity intensity period, and a low activity intensity period, performing first monitoring that detects a declining trend in the heart rate of the exerciser, in the low activity intensity period each time, and monitors whether or not the declining trend of this time is low as compared with a predetermined reference, performing second monitoring that, in a case in which the monitoring by the first monitoring is affirmed, monitors whether or not the activity intensity to be detected in the high activity intensity period of a next time is low as compared with activity intensities detected in high activity intensity periods up to a previous time, and, in a case in which a result of the second monitoring is affirmed, forecasting an outbreak of disease.

Moreover, a non-transitory computer readable storage medium storing a program that causes a computer to forecast an outbreak of disease, the forecasting includes detecting an activity intensity, a high activity intensity period, and a low activity intensity period, from a movement of an exerciser that is detected by an exercise sensor that continuously detects the movement of the exerciser accompanied by intermittent exercise, performing first monitoring that detects a declining trend in a heart rate of the exerciser by a heart rate meter that continuously measures the heart rate of the exerciser, in the low activity intensity period each time, and monitors whether or not the declining trend of this time is low as compared with a predetermined reference, performing second monitoring that, in a case in which the monitoring by the first monitoring is affirmed, monitors whether or not the activity intensity to be detected in the high activity intensity period of a next time is low as compared with activity intensities detected in high activity intensity periods up to a previous time, and, in a case in which a result of the second monitoring is affirmed, forecasting an outbreak of disease.

According to these inventions, a risk of disease outbreak in a first stage is managed by monitoring the declining trend of the heart rate in the low activity intensity period, and, in response to the risk of disease outbreak in the first stage, a risk of disease outbreak in a second stage is managed by monitoring whether a sufficiently high activity intensity in the high activity intensity period, which enables fatigue, heat stroke, and cardiac activity to be forecasted with much higher accuracy. Cardiac disease and heat stroke may be caused by exercise in which relatively high activity intensity continues, and the present invention can forecast such a fatigued state. In this way, biological information to be obtained by sensing and medical knowledge are integrated to detect a state before possibly leading to outbreak of disease, which makes it possible to prevent the outbreak of disease.

### Advantageous Effects of the Disclosure

According to the present invention, biological information and medical knowledge are integrated, which enables an outbreak of disease of an exerciser during exercise to be forecast with high accuracy.

### BRIEF DESCRIPTION OF DRAWINGS

FIG. 1 is an overall configuration diagram showing an embodiment of a disease outbreak forecasting apparatus according to the present invention.

FIG. 2A is part of a flow chart showing a procedure of disease outbreak forecasting processing I.

FIG. 2B is remaining part of the flow chart showing the procedure of disease outbreak forecasting processing I.

FIG. 3A is part of a flow chart showing a procedure of disease outbreak forecasting processing II.

FIG. 3B is remaining part of the flow chart showing the procedure of disease outbreak forecasting processing II.

FIG. 4 is a time chart showing a result of an example of an experiment, and Signal (A) shows activity intensity, Signal (B) shows a heart rate, Signal (C) shows an eardrum temperature, and Signal (D) shows a heat index (WBGT: Wet-Bulb Globe Temperature).

### DESCRIPTION OF EMBODIMENTS

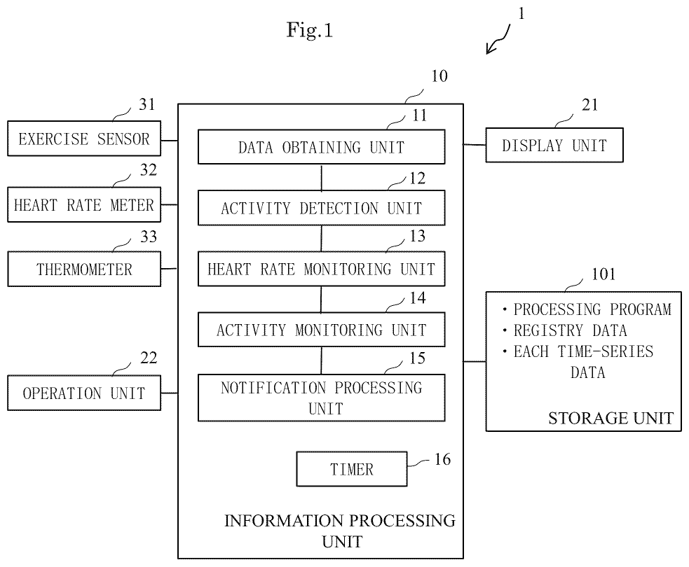

FIG. 1 is an overall configuration diagram showing an embodiment of a disease outbreak forecasting apparatus. In FIG. 1, the disease outbreak forecasting apparatus 1 includes an information processing unit 10 preferably configured by a computer (a processor). The information processing unit 10 is connected to a storage unit 101, a display unit 21 that displays an image when necessary, and an operation unit 22. In addition, the information processing unit 10 is connected to various sensors to collect biological information of an exerciser through a typically short-range type wireless communication means. In the present embodiment, an exercise sensor 31, a heart rate meter 32, and a thermometer 33 that measures temperature of exercise environment when necessary.

The storage unit 101 has a processing program for disease outbreak forecasting processing, a memory area that stores various types of registry data or the like to be referred to when the processing program is executed, and a work area that temporarily stores data (time series data obtained from outside, arithmetic processing data, or the like) being processed. The display unit 21 displays result information after information processing or performs check display of content of an operation input. The operation unit 22 is an input device provided with a mouse and a keyboard. It is to be noted that the operation unit 22 may employ a so-called publicly known touch panel in which a transparent sheet made of a pressure-sensitive element is stacked on a screen of the display unit 21 to receive instructions to a corresponding button by pressing on a displayed button or icon.

The exercise sensor 31 can apply to an accelerometer or a GPS (Global Positioning System) receiver that is attached to a part of body or a proper place of clothes. In addition, as the exercise sensor 31, an installed type video camera (an imaging device) that captures an exercise region of an exerciser may be used, and a biological activity (a movement) of a target exerciser may be detected from a video image of the video camera. For example, a publicly known skeleton detection algorithm (Open Pose) is employable. This method, even in a case of a plurality of exercisers, detects the movement of a waist position, for example, by extracting a feature point (a joint position: node) and a feature direction (a framework: skeleton) from a video of each labeled player.

The heart rate meter 32 may be a wearable sensor such as a biological body attached type including a wristwatch type, or a wear type, and monitors a heart rate during exercise or sporting activity.

In addition, the thermometer 33 detects the body temperature of an exerciser, and, in the present embodiment, is applicable to an ear thermometer that is inserted into the ear of the exerciser and measures eardrum temperature considered to be close to brain temperature. It is to be noted that, as the thermometer 33, the exerciser may be substituted by the temperature of the exercise environment. According to the temperature detected by the thermometer 33, the setting of a predetermined value α (see Signal (A)) as a monitoring reference, in response to temperature characteristics of the heart rate, is able to be changed, and higher accurate forecasting is achievable.

In the present embodiment, for example, the exercise sensor 31, the heart rate meter 32, and the thermometer 33 are attached to the exerciser, and, on the other hand, various aspects are employable for the placement configuration of the information processing unit 10 (including the storage unit 101), the display unit 21, and the operation unit 22. In the present embodiment, the information processing unit 10 is configured by a portable personal computer, includes the display unit 21 and the operation unit 22, and will be described below as an aspect in which an administrator operates.

Therefore, in the present embodiment, biological information as an index is received by the information processing unit 10, from a device that is attached to the exerciser during exercise and detects various types of biological information, and the information processing unit 10 continuously executes processing of disease outbreak forecasting based on various types of received biological information, and makes output to the display unit 21. It is to be noted that a method of notifying forecasting may be a sound and a predetermined alarm, for example, in addition to an image. In addition to the image, especially in a case of acoustics, a function unit that wirelessly receives a forecasting signal from near the information processing unit 10 and outputs as an acoustic signal may be provided in any of the sensors on a side of the exerciser. It is to be noted that other aspects will be described below.

In FIG. 1, the information processing unit 10, by reading a processing program stored in the storage unit 101 and executing the program on a main memory (a work area), functions as the data obtaining unit 11, the activity detection unit 12, the heart rate monitoring unit 13, the activity monitoring unit 14, the notification processing unit 15, and the timer 16.

The data obtaining unit 11 continuously captures a biological signal in a predetermined period, from the exercise sensor 31, the heart rate meter 32, and the thermometer 33. An activity intensity signal from the exercise sensor 31 is preferably the magnitude of acceleration. A heart beat signal or a heart rate signal from the heart rate meter 32 is converted and is outputted as the number of heart beats/minute. A temperature signal from the thermometer 33 is captured in a relatively long period in the present embodiment, for example, every several minutes. For example, the time chart of Signals (A) and (B) is shown as an example of an experiment.

The activity detection unit 12 executes processing to binarize the activity intensity in a level direction, from the activity intensity signal detected by the exercise sensor 31. For example, by using a predetermined level, or a level around a median value of the activity intensity according to a type of exercise, or the like, as a threshold, an exercise period is divided into a high activity intensity period and a low activity intensity period. As illustrated in Signal (A), intermittent exercise is also performed in a temporal direction.

The present embodiment forecasts a state that can lead to an outbreak of a serious disease such as a heart disease, heat stroke, or fatigue, during sporting activity, from a time-series change in an activity level and a heart rate in intermittent physical exercise between a period of relatively high activity intensity and a period of relatively low activity intensity, during exercise or sporting activity. The intermittent physical exercise is not a constant activity level but is physical activity in exercise or sport in which the relatively high activity intensity and the relatively low activity intensity (also including activity and rest (break)) alternate, and is seen in many sports such as circuit training, tennis, soccer, baseball, basketball, volleyball, or boxing, for example.

The reason to divide the exercise period into the high activity intensity period and the low activity intensity period is that, in a case in which a heart rate during exercise is detected, although a method of electrically monitoring the function of a heart as an electrocardiogram by attaching an electrode to a body surface or sensing a pulse rate by use of a pulse pressure or a light beam is possible, it is difficult to monitor electrocardio or a pulse rate with high accuracy, during activity. Then, in the present embodiment, as described below, a heart rate is detected with high accuracy at timing of a relatively small activity level in intermittent exercise.

The heart rate monitoring unit 13 extracts a heart rate obtained after a predetermined time from a start time point of the low activity intensity period each time. The timer 16 times this predetermined time. In addition, the predetermined time is able to be appropriately set up according to the type or the like of exercise or sport, and, in a case in which a rest period is set up in advance in the middle of the exercise, for example, may be at an intermediate time point or an end time point of set time. Extracting a heart rate within the low activity intensity period is able to obtain highly accurate biological information.

Moreover, the heart rate monitoring unit 13 detects a declining trend in the heart rate of the exerciser, and monitors whether or not the declining trend of this time is low as compared with a predetermined reference. As the declining trend, the heart rate itself may be used as an index. In this case, whether the heart rate extracted this time is larger than a value obtained by adding the predetermined value a to the heart rate up to the previous time, as the predetermined reference, is monitored. Herein, the heart rate up to the previous time may be the heart rate extracted at the previous time or the average of heart rates extracted until the previous time, or may be the first heart rate at start of monitoring. Further, the predetermined value α is able to be set to an appropriate value (as a dead zone) according to the type or the like of exercise or sport, and may be considered as a value “5” or the like, for example. In this case, in a case in which the predetermined reference is set to “100,” the heart rate monitoring unit 13 determines whether or not the heart rate of this time is larger than “105” (=100+5). This determination is positioned as a first stage of a risk of disease outbreak.

The activity monitoring unit 14, in a case in which a monitoring determination by the heart rate monitoring unit 13 is affirmed, subsequently makes a determination as a second stage of the risk of disease outbreak in terms of activity intensity. The activity monitoring unit 14 monitors whether or not the activity intensity to be detected, immediately after the monitoring determination by the heart rate monitoring unit 13 is affirmed, that is, in the high activity intensity period of a next time is low as compared with activity intensities detected in high activity intensity periods up to a previous time. Specifically, the activity monitoring unit 14 monitors whether or not the activity intensity to be detected in the high activity intensity period of the next time is lower by a predetermined difference β or more than the activity intensity detected in the high activity intensity periods up to the previous time.

Herein, the activity intensity detected in the high activity intensity periods up to the previous time may be the activity intensity detected at the previous time or the average of activity intensities detected until the previous time, or may be the first activity intensity or the average value. In addition, the average value may be replaced with a peak value. Moreover, the predetermined difference β as the dead zone may be appropriately set up according to the type of exercise or sport. A state in which the activity intensity during exercise is reduced is detected as an at-risk state in the second stage, which is an even higher risk due to continuous exercise.

In this way, the risk is evaluated in two stages with the application of different indexes for different periods, so that heat stroke, extreme fatigue, or the like is able to be effectively prevented with high accuracy from occurring.

The notification processing unit 15, in a case in which the activity intensity to be detected in the high activity intensity period of the next time is determined to be lower by the predetermined difference β or more than the activity intensity detected in the high activity intensity periods up to the previous time, by the activity monitoring unit 14, outputs forecasting notification noting that the risk of disease outbreak is high, to the display unit 21, for example.

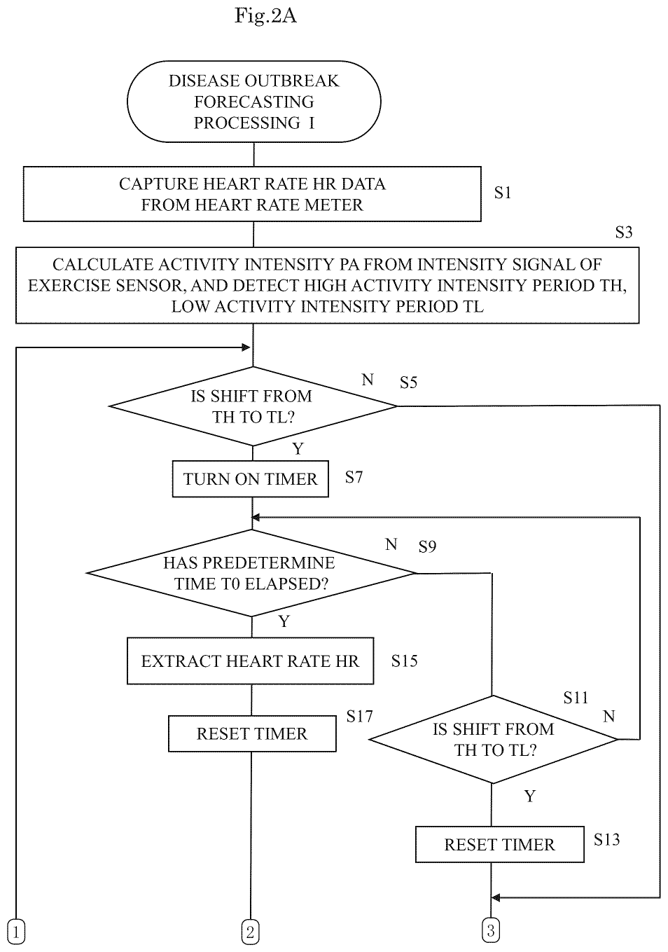

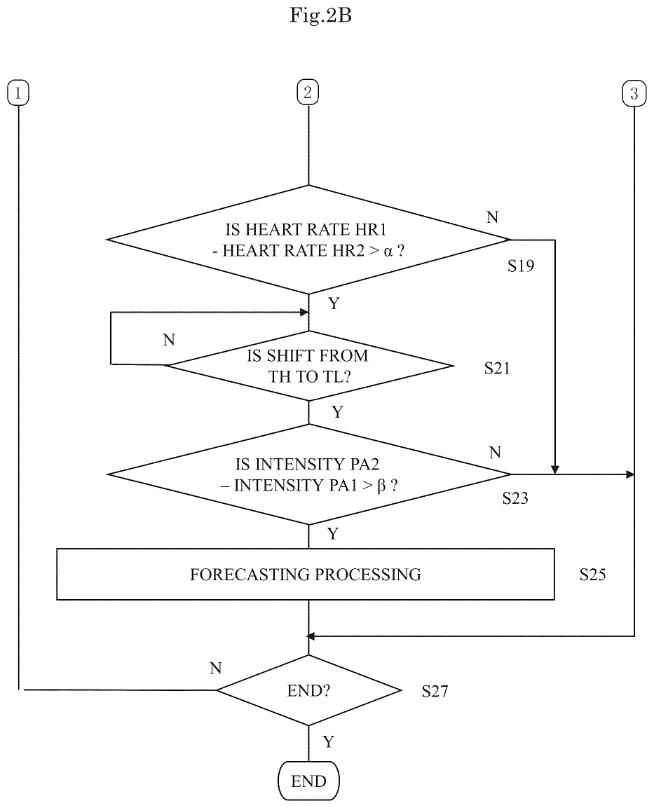

FIG. 2A and FIG. 2B are flow charts showing a procedure of disease outbreak forecasting processing I. First, when a processing program starts, capturing heart rate HR data from the heart rate meter 32 is started (Step S1), and calculation of the activity intensity PA from an intensity signal to be captured together from the exercise sensor 31 and detection of the high activity intensity period TH and the low activity intensity period TL are also performed (Step S3).

Subsequently, whether or not the intensity signal is shifted from the high activity intensity period TH to the low activity intensity period TL is determined (Step S5). In a case in which it is determined that the intensity signal has been shifted, a timer that times a predetermined time TO is turned on (ON) (Step S7), whereas, in a case in which it is determined that the activity signal has not been shifted, subsequently, whether or not the processing program ends is determined (Step S27), and, when the processing program ends, this flow exits, and, when the processing program does not end, the processing returns to Step S5 and the same processing is repeated.

In the case in which the timer is turned on in Step S7, whether the predetermined time TO has elapsed is subsequently determined (Step S9). When it is before the predetermined time TO elapses, whether the activity has shifted from the low activity intensity period TL to the high activity intensity period TH is subsequently determined (Step S11), and, in a case in which it is determined that the activity has not shifted, the processing returns to Step S9 and timing continues, whereas, in a case in which it is determined that the activity has shifted, it is determined that the low activity intensity period TL is too short, the timer is reset (Step S13), and the processing proceeds to Step S27.

On the other hand, in the case in which the predetermined time TO has elapsed in Step S9, the heart rate HR is extracted (Step S15), and subsequently the timer is reset (Step S17).

Next, whether or not a heart rate HR1 of this time is larger than a value obtained by adding the predetermined value a to a heart rate HR2 up to the previous time is determined (Step S19). In a case in which this determination is affirmed, whether the activity has shifted from the low activity intensity period TL to the high activity intensity period TH is subsequently determined (Step S21), and, after the shift is waited, whether or not an activity intensity PA1 to be detected in the high activity intensity period TH of the next time is lower by the predetermined difference β or more than an activity intensity PA2 to be detected in the high activity intensity periods TH up to the previous time is determined (Step S23). In a case in which the determination is affirmed in Step S23, forecasting processing to notify the risk of disease outbreak is executed (Step S25), and the processing proceeds to Step S27. On the other hand, in a case in which the determination in Step S19 and Step S23 is denied, the processing proceeds to Step S27 as it is.

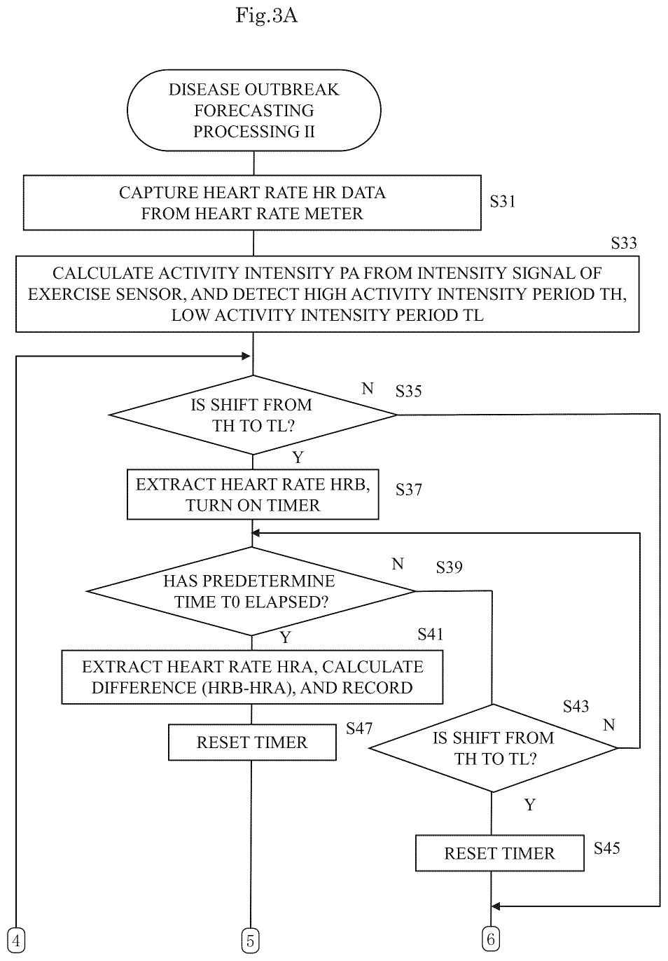

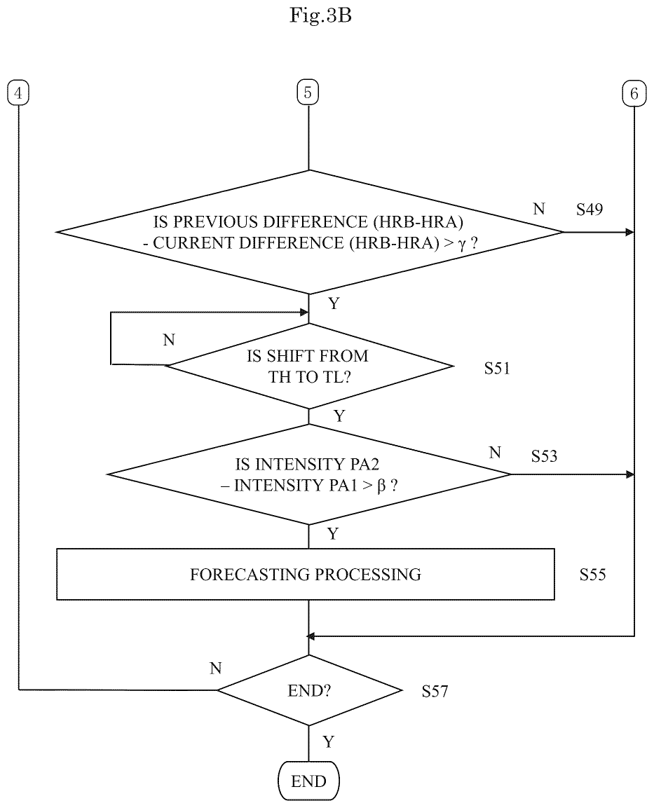

Next, FIG. 3A and FIG. 3B are flow charts showing a procedure of disease outbreak forecasting processing II. The disease outbreak forecasting processing II, while the disease outbreak forecasting processing I monitors the change in the heart rate HR at a time point when the predetermined time elapses from the start of the low activity intensity period TL, monitors a change (in other words, a declining trend) in the difference between the heart rate HR1 at the start time point of the low activity intensity period TL and the heart rate HR2 at the time point when the predetermined time elapses. The procedure of the disease outbreak forecasting processing II is different in Step S37, Step S41, and Step S49 from the procedure of the disease outbreak forecasting processing I, and these will be described below.

Step S37 extracts a heart rate HRb and turns on the timer, at a time point when the activity shifts from the low activity intensity period TL to the high activity intensity period TH.

Step S41, after the shift to the low activity intensity period TL, extracts a heart rate HRa and calculates and records a difference between the two (HRb−HRa) at the time point when the predetermined time TO elapses.

Step S49 monitors the declining trend of the heart rate and determines whether the difference between the difference (HRb−HRa) of the previous time and the difference (HRb−HRa) of this time is larger than a predetermined value γ. In a case in which this determination is affirmed, the declining trend of the heart rate is lower than the reference, and a state is determined to be at risk in the first stage.

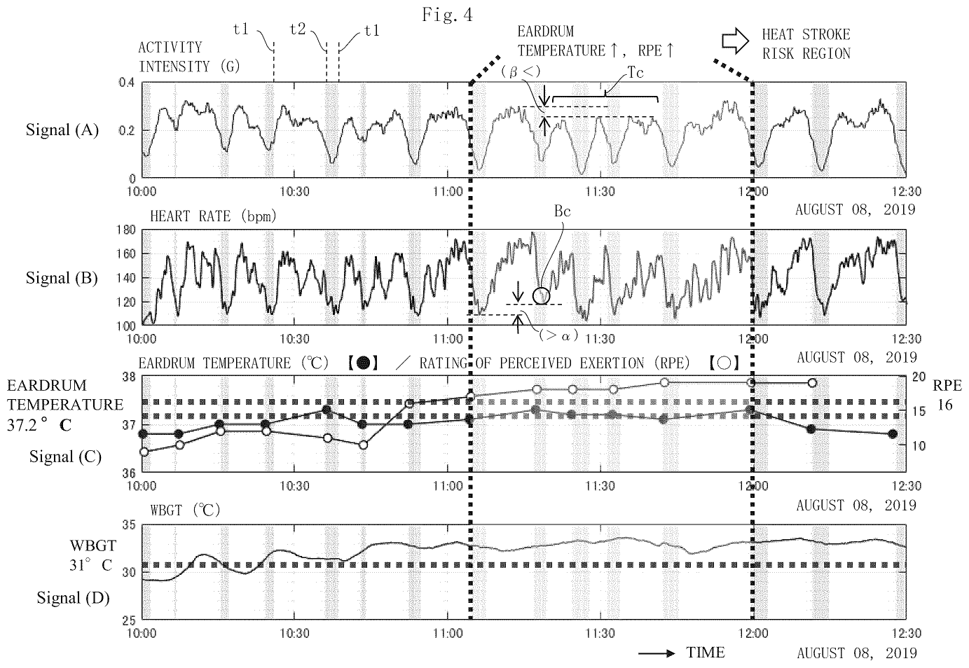

FIG. 4 is a time chart showing a result of an example of an experiment, and Signal (A) shows activity intensity, Signal (B) shows a heart rate, Signal (C) shows an eardrum temperature, and Signal (D) shows a heat index (WBGT: Wet-Bulb Globe Temperature). This example of the experiment was conducted with college students as subjects in a simulated tennis game, and, as shown in Signal (A), the exercise was performed from 10:00 to 12:30. In addition, the low activity intensity period (a section between time points t2 and t1 in each time, see Signal (A)) corresponds to a set break (for two minutes for every end of a set), and a coat change break (90 seconds).

Moreover, a wearable sensor as the exercise sensor 31 and a DSP wireless ECG/HR logger (produced by sports sensing company) as the heart rate meter 32 were used. Further, In FIG. 4, as a reference, an ear thermometer MC-510 named Ken-on-kun Mimi (produced by OMRON Corporation) was used as an eardrum temperature, and the heat index (wet-bulb globe temperature: WBGT) and Rating of Perceived Exertion (RPE) was expressed by the Borg scale.

The intermittent exercise in the high activity intensity period (t1 to t2) during exercise and the low activity intensity period (t2 to t1) during a break is repeatedly appeared (Signal (A)).

The heart rate heavily fluctuates up and down in the high activity intensity period (t1 to t2) during exercise, while, when the shift is made to the low activity intensity period (t2 to t1), gradually reducing in the same declining trend each time, and, when the shift is made to activity after the break ends, turns upward again. The heart rate can be seen to drop to around “110” during the break. On the other hand, in the break around 11:20, the heart rate drops only down to “120” (see Bc in Signal (B) (difference >α)). Here, first stage risk outbreak forecasting was issued.

Furthermore, with respect to this time point Bc, the activity intensity of at least a next section (a next time) is lower by the predetermined value β or more than a value of the previous activity intensities, for example, peak intensity (see the Tc period in Signal (A), (β<difference)), and the state in which the movement may be slow due to fatigue, for example, was considered, and, here, second stage risk outbreak forecasting was issued. It is to be noted that, in the experiment, after the first stage risk outbreak, measures such as fully cooling a body surface during a rest period were taken, so that the exercise was able to be subsequently continued.

It is to be also noted that, in Signals (C) and (D) in FIG. 4, the eardrum temperature during the exercise period was around 37° C., and the Rating of Perceived Exertion was about “15” to about “19,” and the WBGT was a little over “31”. The content of the rating of perceived exertion is shown in (Table 1).

TABLE 1
RATING OF PERCEIVED EXERTION

| | |
|---|---|
| 6 | EXTREMELY LIGHT |
| 7 | |
| 8 | |
| 9 | VERY LIGHT |
| 10 | |
| 11 | LIGHT |
| 12 | |
| 13 | SOMEWHAT HARD |
| 14 | |
| 15 | HARD |
| 16 | |
| 17 | VERY HARD |
| 18 | |
| 19 | EXTREMELY HARD |
| 20 | |

It is to be noted that, although the present embodiment describes the information processing unit 10 as a portable type personal computer that a manager can manage, an exerciser may directly recognize a risk of disease outbreak. For example, the heart rate meter 32 may include a function of the information processing unit 10, the display unit 21, or an acoustic unit.

In addition, in the present embodiment, the timing of extracting a heart rate, although having been set after a predetermined time elapses in the low activity intensity period, is not limited to this, and, for example, may always be at an end time point of the low activity intensity period. In this case, processing to convert the extracted heart rate into a value for the predetermined time, from the time to the end time point of the low activity intensity period and the drop characteristics of the heart rate prepared in advance may be executed. According to this method, the heart rate is able to be extracted each time, regardless of the length of the low activity intensity period.

Moreover, the exercise in the present invention, in addition to exercises defined by known predetermined rules, may include self-taught jogging, walking, and other various types of training and sports that are accompanied by intermittent exercise in general.

As described above, the disease outbreak forecasting apparatus according to the present invention preferably includes an exercise sensor that continuously detects a movement of an exerciser accompanied by intermittent exercise, a heart rate meter that continuously measures a heart rate of the exerciser, and an activity detection unit that detects, from the movement of the exerciser that is detected by the exercise sensor, an activity intensity, a high activity intensity period, and a low activity intensity period, a first monitoring unit that detects a declining trend in the heart rate of the exerciser, in the low activity intensity period each time, and monitors whether or not the declining trend of this time is low as compared with a predetermined reference, a second monitoring unit that, in a case in which the monitoring by the first monitoring unit is affirmed, monitors whether or not the activity intensity to be detected in the high activity intensity period of a next time is low as compared with activity intensities detected in high activity intensity periods up to a previous time, and a notifying unit that, in a case in which the monitoring by the second monitoring means is affirmed, forecasts an outbreak of disease.

In addition, a disease outbreak forecasting method according to the present invention preferably includes continuously detecting a movement of an exerciser accompanied by intermittent exercise by an exercise sensor, and continuously measuring a heart rate of the exerciser by a heart rate meter, preferably detecting, from the movement of the exerciser that is detected by the exercise sensor, an activity intensity, a high activity intensity period, and a low activity intensity period, performing first monitoring that preferably detects a declining trend in the heart rate of the exerciser, in the low activity intensity period each time, and preferably monitors whether or not the declining trend of this time is low as compared with a predetermined reference, performing second monitoring that, in a case in which the monitoring by the first monitoring is affirmed, preferably monitors whether or not the activity intensity to be detected in the high activity intensity period of a next time is low as compared with activity intensities detected in high activity intensity periods up to a previous time, and, in a case in which the monitoring by the second monitoring is affirmed, preferably forecasting an outbreak of disease.

Moreover, a non-transitory computer readable storage medium storing a program that causes a computer to forecast an outbreak of disease, the forecasting includes detecting an activity intensity, a high activity intensity period, and a low activity intensity period, from a movement of an exerciser that is detected by an exercise sensor that continuously detects the movement of the exerciser accompanied by intermittent exercise, performing first monitoring that detects a declining trend in a heart rate of the exerciser by a heart rate meter that continuously measures the heart rate of the exerciser, in the low activity intensity period each time, and monitors whether or not the declining trend of this time is low as compared with a predetermined reference, performing second monitoring that, in a case in which the monitoring by the first monitoring is affirmed, monitors whether or not the activity intensity to be detected in the high activity intensity period of a next time is low as compared with activity intensities detected in high activity intensity periods up to a previous time, and, in a case in which a result of the second monitoring is affirmed, forecasting an outbreak of disease.

According to these inventions, a risk of disease outbreak in the first stage is managed by monitoring the declining trend of the heart rate in the low activity intensity period, and, in response to the risk of disease outbreak in the first stage, a risk of disease outbreak in the second stage is managed by monitoring whether a sufficiently high activity intensity in the high activity intensity period, which enables fatigue, heat stroke, and cardiac activity to be forecasted with much higher accuracy. Cardiac disease and heat stroke may be caused by exercise in which relatively high activity intensity continues, and the present invention can forecast such a fatigued state. In this way, biological information to be obtained by sensing and medical knowledge are integrated to detect a state before possibly leading to outbreak of disease, which makes it possible to prevent the outbreak of disease.

In addition, the first monitoring means preferably detects the heart rate as the declining trend of the heart rate every time point when a predetermined time elapses in the low activity intensity period each time, and preferably monitors whether or not the heart rate of this time is larger than a value obtained by adding a predetermined value to the heart rates up to the previous time, the value being the predetermined reference. According to this configuration, the first monitoring means enables monitoring in the first stage by use of the heart rate every time point when a predetermined time elapses is achieved.

Moreover, the heart rates up to the previous time preferably include at least one heart rate in the low activity intensity period up to the previous time. According to this configuration, it is easy to obtain the heart rate up to the previous time used as a reference.

In addition, the heart rates up to the previous time are preferably heart rates in the low activity intensity period at the first time. According to this configuration, as a heart rate used as a reference, a value closer to a calm state of the exerciser is employed.

Moreover, the first monitoring means preferably detects a first heart rate at a start time point in the low activity intensity period each time, and a second heart rate at a time point when a predetermined time elapses in the low activity intensity period that follows immediately, and preferably monitors whether or not a current difference being a difference between the first heart rate of this time and the second heart rate of this time as the declining trend of the heart rate is smaller than a value obtained by subtracting a predetermined value from a previous difference being a difference between first heart rates and second heart rates up to the previous time, the value being the predetermined reference. According to this configuration, the change in the amount of reduction in the heart rate for each low activity intensity period is compared in size to the reference.

In addition, the first heart rates and the second heart rates up to the previous time preferably include at least one heart rate in the low activity intensity period up to the previous time. According to this configuration, it is easy to obtain the first heart rates and the second heart rates up to the previous time on a reference side.

Moreover, the second monitoring means preferably monitors whether or not the activity intensity to be detected in the high activity intensity period of the next time is lower by a predetermined difference or more than the activity intensity detected in the high activity intensity periods up to the previous time. According to this configuration, it is monitored that the activity intensity in the high activity intensity period is low by the predetermined difference or more, that is, that sufficiently high activity intensity is not obtained.

In addition, the present invention includes a thermometer that detects a temperature of an exercise environment, and the first monitoring means preferably changes the predetermined reference according to a detected temperature. According to this configuration, higher accurate forecasting is achievable in response to the temperature characteristics of the heart rate. It is to be noted that the temperature of the exercise environment may include the periphery of an exerciser and the body temperature of the exerciser.

### REFERENCE SIGNS LIST

1 disease outbreak forecasting apparatus
10 information processing unit
101 storage unit
12 activity detection unit
13 heart rate monitoring unit (first monitoring means)
14 activity monitoring unit (second monitoring means)
15 notification processing unit (a part of notifying means)
21 display unit (a part of notifying means)
31 exercise sensor
32 heart rate meter
33 thermometer

1. A disease outbreak forecasting apparatus comprising:
an exercise sensor that continuously detects a movement of an exerciser accompanied by intermittent exercise;
a heart rate meter that continuously measures a heart rate of the exerciser;
an activity detection means that detects, from the movement of the exerciser that is detected by the exercise sensor, an activity intensity, a high activity intensity period, and a low activity intensity period;
a first monitoring means that detects a declining trend in the heart rate of the exerciser, in the low activity intensity period each time, and monitors whether or not the declining trend of this time is low as compared with a predetermined reference;
a second monitoring means that, in a case in which the monitoring by the first monitoring means is affirmed, monitors whether or not the activity intensity to be detected in the high activity intensity period of a next time is low as compared with activity intensities detected in high activity intensity periods up to a previous time; and
a notifying means that, in a case in which the monitoring by the second monitoring means is affirmed, forecasts an outbreak of disease.

2. The disease outbreak forecasting apparatus during exercise according to claim 1, wherein the first monitoring means detects the heart rate as the declining trend of the heart rate every time point when a predetermined time elapses in the low activity intensity period each time, and monitors whether or not the heart rate of this time is larger than a value obtained by adding a predetermined value to heart rates up to the previous time, the value being the predetermined reference.

3. The disease outbreak forecasting apparatus according to claim 2, wherein the heart rates up to the previous time include at least one heart rate in the low activity intensity period up to the previous time.

4. The disease outbreak forecasting apparatus according to claim 3, wherein the heart rates up to the previous time are heart rates in the low activity intensity period at a first time.

5. The disease outbreak forecasting apparatus according to claim 1, wherein the first monitoring means detects a first heart rate at a start time point in the low activity intensity period each time, and a second heart rate at a time point when a predetermined time elapses in the low activity intensity period that follows immediately, and monitors whether or not a current difference being a difference between the first heart rate of this time and the second heart rate of this time as the declining trend of the heart rate is smaller than a value obtained by subtracting a predetermined value from a previous difference being a difference between first heart rates and second heart rates up to the previous time, the value being the predetermined reference.

6. The disease outbreak forecasting apparatus according to claim 5, wherein the first heart rates and the second heart rates up to the previous time include at least one heart rate in the low activity intensity period up to the previous time.

7. The disease outbreak forecasting apparatus during exercise according to any one of claims 1 to 6, wherein the second monitoring means monitors whether or not the activity intensity to be detected in the high activity intensity period of the next time is lower by a predetermined difference or more than the activity intensity detected in the high activity intensity periods up to the previous time.

8. The disease outbreak forecasting apparatus during exercise according to any one of claims 1 to 7, further comprising a thermometer that detects a temperature of an exercise environment, wherein the first monitoring means changes the predetermined reference according to a detected temperature.

9. A disease outbreak forecasting method comprising using an exercise sensor that continuously detects a movement of an exerciser accompanied by intermittent exercise, and a heart rate meter that continuously measures a heart rate of the exerciser, wherein:
an activity detection means detects, from the movement of the exerciser that is detected by the exercise sensor, an activity intensity, a high activity intensity period, and a low activity intensity period;
a first monitoring means detects a declining trend in the heart rate of the exerciser, in the low activity intensity period each time, and monitors whether or not the declining trend of this time is low as compared with a predetermined reference;
a second monitoring means, in a case in which the monitoring by the first monitoring means is affirmed, monitors whether or not the activity intensity to be detected in the high activity intensity period of a next time is low as compared with activity intensities detected in high activity intensity periods up to a previous time; and
a notifying means, in a case in which the monitoring by the second monitoring means is affirmed, forecasts an outbreak of disease.

10. A program that causes a computer to function as the disease outbreak forecasting apparatus according to any one of claims 1 to 8.
